(19) United States
(12) Patent Application Publication
(10) Pub. No.: US 2008/0281159 A1
(43) Pub. Date: Nov. 13, 2008

(54) Coordinating image acquisition among multiple endoscopes

(75) Inventors: Richard S. Johnston, Sammamush, WA (US); Satoshi Karasawa, Tokyo (JP)

Correspondence Address: BLAKELY SOKOLOFF TAYLOR & ZAFMAN LLP, 1279 OAKMEAD PARKWAY, SUNNYVALE, CA, 94085-4040, US

(73) Assignees: University of Washington; PENTAX Corporation

(21) Appl. No.: 11/801,219

(22) Filed: May 8, 2007

(51) Int. Cl. A61B 1/00

(52) U.S. Cl. 600/160

(57) ABSTRACT

Coordinating image acquisition among multiple endoscopes is disclosed. A method may include acquiring an image of a surface with a first endoscope system by illuminating the surface with light, collecting backscattered light, and generating the image based on the collected backscattered light. An intensity of the light from the first endoscope system may be reduced. While the intensity of the light is reduced, an image of the surface may be acquired with a second, scanning beam endoscope system by scanning a beam of light over the surface and collecting backscattered light at different times while the beam is scanned over the surface. In one aspect, a coordination signal may be exchanged between the endoscope systems to coordinate the image acquisition. In another aspect, the image acquisition may be coordinated through the scanning beam endoscope system detecting the intensity reduction. Apparatus useful in performing such methods are also disclosed.

### BACKGROUND

1. Field

Embodiments of the invention relate to endoscopes. In particular, embodiments of the invention relate to coordinating image acquisition among multiple endoscopes.

2. Background Information

Endoscopes are instruments or devices that may be inserted into a patient and used to look inside a body cavity, lumen, or otherwise look inside the patient.

One type of endoscope is a scanning beam endoscope. The scanning beam endoscope may scan a beam or illumination spot over a surface to be viewed. Backscattered light from the illumination spot may be detected by the scanning beam endoscope at different times during the scan in order to construct an image of the surface.

Another type of endoscope is a conventional, non-scanning beam endoscope. Such endoscopes may flood the whole surface to be viewed with a bright white or near white light, for example, provided through one or more generally large multimode optical fibers. Backscattered light may be collected from the whole surface in parallel, and an image may be constructed. In some such endoscopes, a light detector array, for example a charge-coupled device (CCD), may be included at a distal tip of the endoscope to detect the backscattered light. In other endoscopes, numerous optical fibers, each corresponding to a pixel in the image, may be used to collect and return the backscattered light to a base station. In the base station, the light may be detected with a light detector array, or otherwise used to construct the image.

Multiple endoscopes are occasionally used in combination. By way of example, a so-called mother endoscope may be used with a so-called daughter or baby endoscope. By way of example, the daughter or baby scope may be used to view areas beyond the reach of the mother endoscope.

### BRIEF DESCRIPTION OF THE SEVERAL VIEWS OF THE DRAWINGS

The invention may best be understood by referring to the following description and accompanying drawings that are used to illustrate embodiments of the invention. In the drawings:

FIG. 1 is a block flow diagram of a method of coordinating imaging in a dual endoscope system that includes acquiring an image of a surface with a scanning beam endoscope system while an intensity of light from another associated endoscope system is reduced, according to embodiments of the invention.

FIG. 2 is a block flow diagram of a method of coordinating image acquisition of a scanning beam endoscope system based on the exchange of a coordination signal, according to embodiments of the invention.

FIG. 3 is a block diagram of one example of a dual endoscope system that includes a communication link between endoscope systems to exchange synchronization or coordination signals in order to synchronize or coordinate image acquisition, according to embodiments of the invention.

FIG. 4 is a block flow diagram of a method of coordinating image acquisition of a scanning beam endoscope system by detecting reduction of intensity of light from another endoscope system, according to embodiments of the invention.

FIG. 5 is a block diagram of one example of a scanning beam endoscope base station having at least one optional photodetector and a controller to coordinate image acquisition with another endoscope system by detecting a reduction of intensity of the light from the other endoscope system, according to embodiments of the invention.

### DETAILED DESCRIPTION

In the following description, numerous specific details are set forth. However, it is understood that embodiments of the invention may be practiced without these specific details. In other instances, well-known circuits, structures and techniques have not been shown in detail in order not to obscure the understanding of this description.

Challenges may present themselves when certain types of endoscopes, such as, for example scanning beam endoscopes, are used in combination with other endoscopes, such as, for example, the aforementioned conventional endoscopes. For example, if the surface over which the beam or illumination spot from the scanning beam endoscope is scanned is concurrently illuminated with light from another endoscope, then some of the light from the other endoscope may be reflected or otherwise backscattered and collected by the scanning beam endoscope. This light from the other endoscope is generally unwanted and may represent noise, which may tend to reduce the contrast or otherwise adversely affect the quality of images acquired using the scanning beam endoscope.

The inventors have conceived of improved systems and methods of imaging in multiple endoscope systems. In embodiments of the invention, a first endoscope and a second scanning beam endoscope may be synchronized or otherwise coordinated so that an intensity of light from the first endoscope is turned off, or at least reduced, while the scanning beam endoscope acquires an image of a surface. Advantageously, this may help to improve the contrast or quality of the image acquired using the scanning beam endoscope.

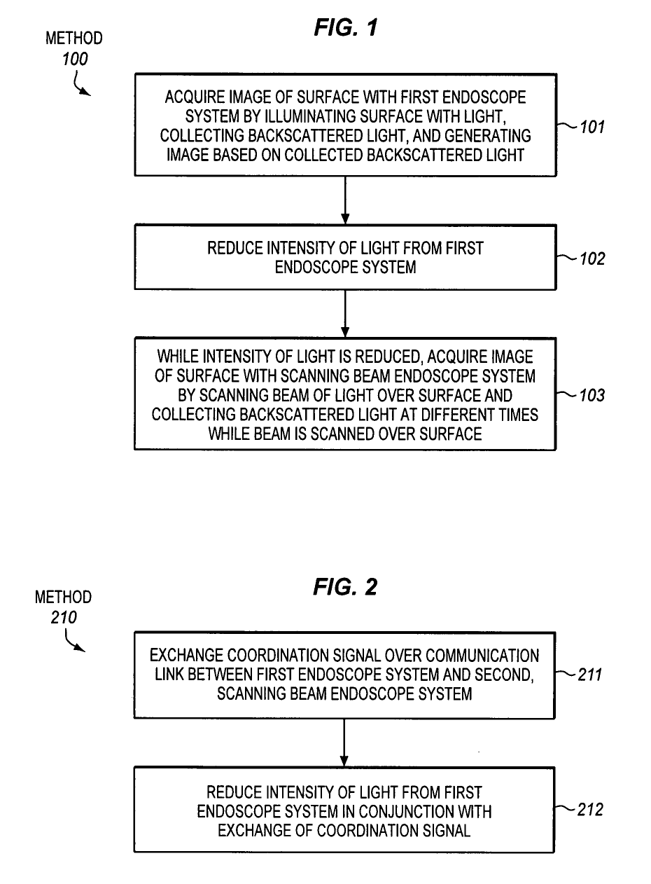

FIG. 1 is a block flow diagram of a method 100 of coordinating imaging in a dual endoscope system that includes acquiring an image of a surface with a scanning beam endoscope system while an intensity of light from another associated endoscope system is reduced, according to embodiments of the invention.

Initially, an image of a surface may be acquired with a first endoscope system, at block 101. In embodiments of the invention, the first endoscope system may be a conventional endoscope system, and acquiring the image may include illuminating the surface with generally broad bandwidth light, collecting backscattered light, and generating the image based on the collected backscattered light.

Then, an intensity of the light from the first endoscope system may be reduced, or in some embodiments turned off, at block 102. In various embodiments of the invention, the intensity may be reduced by at least 30%, at least 50%, at least 80%, at least 90%, or by about 100%.

While the intensity of the light is reduced or turned off an image of the surface may be acquired with the scanning beam endoscope system, at block 103. In embodiments of the invention, acquiring the image may include scanning a beam of light over the surface, and collecting backscattered light at different times, while the beam is scanned over the surface. The backscattered light may be detected and used to construct an image of the surface.

Since the image of the surface is acquired with the scanning beam endoscope system, while the intensity of the light from the first endoscope system is reduced or turned off, less of the light from the first endoscope may be backscattered and collected by the scanning beam endoscope system. This may help to improve contrast or otherwise improve quality of images constructed using the scanning beam endoscope system.

The method described above may be repeated, for example multiple or many times each second, during operation of the dual endoscope system. The rate at which the method is performed may depend in part on the desired frames rate. In one aspect, the method may be repeated continually as long as both endoscope systems are used to image the surface.

Now, different approaches are possible for coordinating image acquisition among the endoscope systems. In one example approach, which will be discussed first, coordination may be achieved through the exchange of coordination or synchronization signals. In another example approach, which will be discussed further below, coordination may be achieved through the scanning beam endoscope system sensing or detecting the reduction of the intensity of the light from the first endoscope system with a photodetector.

FIG. 2 is a block flow diagram of a method 210 of coordinating image acquisition of a scanning beam endoscope system based on the exchange of a coordination signal, according to embodiments of the invention.

At block 211, a coordination signal may be exchanged between a first endoscope system and a scanning beam endoscope system over a communication link. In various embodiments of the invention, the first endoscope system may provide the coordination signal to the scanning beam endoscope system, the first endoscope system may receive the coordination signal from the scanning beam endoscope system, or bi-directional signal exchange may take place.

The coordination signal may be used to coordinate or synchronize image acquisition and/or display by the endoscope systems. The coordination signal is commonly electrical, but may instead optionally be optical or wireless. In one particular embodiment, the coordination signal may include a digital signal consisting of a single bit having a first predetermined value, for example zero, to indicate that the light from the first endoscope system is to be turned off or at least reduced. Alternatively, multiple bits may be included to specify different intensity reductions and/or other information. Analog signals (e.g., voltage levels) and a wide variety of other signals capable of conveying coordination information are also possible.

Then, an intensity of the light from the first endoscope system may be reduced or turned off in conjunction with the exchange of the coordination signal, at block 212. As used herein, reduced “in conjunction” with the exchange of the signal may mean reduced “immediately before” the exchange of the signal and prompting the exchange of the signal, reduced “concurrently” with the exchange of the signal, reduced “immediately after” the exchange of the signal and based on the exchange of the signal, or reduced “responsive to” the exchange of the signal. As one illustrative example, the first endoscope system may reduce the intensity concurrently with, immediately before, or immediately after transmitting the signal. As another illustrative example, the scanning beam endoscope system may transmit the coordination signal and the first endoscope system may reduce the intensity responsive to or immediately after receiving the coordination signal. The scanning beam endoscope system may then acquire an image while the intensity is reduced, as previously described.

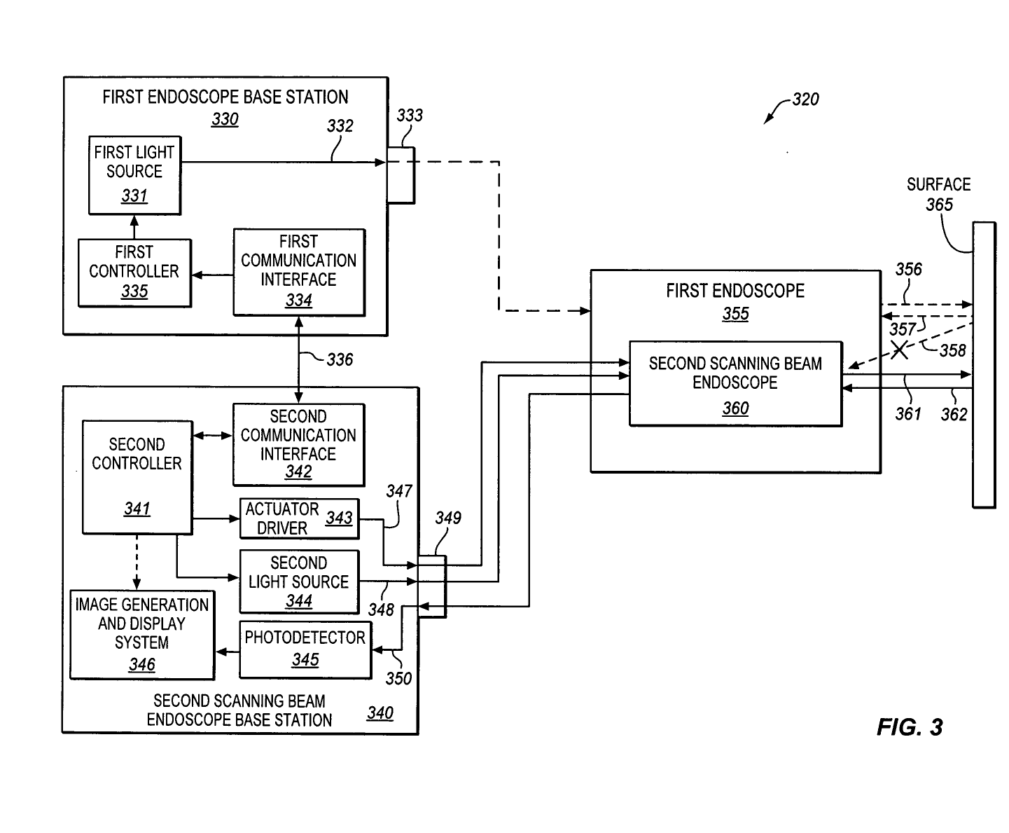

FIG. 3 is a block diagram of one example of a dual endoscope system 320 that includes a communication link 336 between endoscope systems to exchange synchronization or coordination signals in order to synchronize or coordinate image acquisition, according to embodiments of the invention.

The system includes a first endoscope 355, a first endoscope base station 330, a second scanning beam endoscope 360, and a second scanning beam endoscope base station 340. Examples of suitable types of endoscopes include, but are not limited to, bronchoscopes, colonoscopes, gastroscopes, duodenoscopes, sigmoidoscopes, thorascopes, ureteroscopes, sinuscopes, boroscopes, and thorascopes, to name just a few examples. The endoscopes are positioned near a surface 365, of which images are to be acquired.

In the illustration, the first and second endoscopes are arranged or configured as mother and daughter endoscopes, respectively, although this is not required. By way of example, the daughter scope may be inserted or otherwise introduced through an internal working channel of the mother scope prior to, or during use. Alternatively, the second scope may be configured as the mother scope, and the first scope may be configured as the daughter scope. As yet another option, the first and second scopes may simply be used in the same area but not configured as mother and daughter.

The first base station has a first connector interface 333 to allow the first endoscope to be connected. The first base station also has a first light source 331. In embodiments of the invention, the first light source may be a conventional broad bandwidth light source used in conventional endoscopes known in the arts. By way of example, the broad bandwidth light commonly has a bandwidth of at least 200 nanometers (nm). The first light source may provide light 332 to the first endoscope through the first connector interface. The first endoscope may illuminate the surface with light 356. Backscattered light 357 may be collected by the first endoscope and used to construct an image.

The second base station similarly has a second connector interface 349 to allow the scanning beam endoscope to be connected. The second base station also has a second light source 344 to provide light 348 to the scanning beam endoscope through the second connector interface. In embodiments of the invention, the second light source may include at least one, or a plurality, of narrow bandwidth light sources. Examples of suitable narrow bandwidth light sources include, but are not limited to, lasers, laser diodes, vertical cavity surface-emitting lasers (VCSELs), other light emitting devices known in the arts, and combinations thereof. Each narrow bandwidth light source may provide a corresponding narrow bandwidth light to the scanning beam endoscope through the second connector interface. By way of example, each narrow bandwidth light commonly has a bandwidth of less than about 3 nm.

The second base station also includes an actuator driver 343. The actuator driver may provide actuator drive signals 347 to the scanning beam endoscope through the second connection interface. The actuator drive signals may be capable of causing the scanning beam endoscope to scan a beam or illumination spot 361 over the surface in a spiral, propeller, raster, or other scan pattern. In one or more embodiments of the invention, the actuator drive signals may be capable of causing an actuator of the scanning beam endoscope to vibrate a single cantilevered optical fiber close to or within a Q-factor of its resonant frequency, although the scope of the invention is not so limited. Further background information on such scanning, if desired, is available in U.S. Published Application No. 20060138238, entitled “METHODS OF DRIVING A SCANNING BEAM DEVICE TO ACHIEVE HIGH FRAME RATES,” by Richard S. Johnston et al.

The scanning beam endoscope may collect a backscattered portion 362 of the beam or illumination spot. As shown, in one or more embodiments of the invention, the second base station may optionally include at least one photodetector 345 and an image generation and potentially display system 346, although this is not required. Examples of suitable types of photodetectors include, but are not limited to, photodiodes, photomultiplier tubes, phototransistors, other photodetectors known in the arts, and combinations thereof. The photodetector is positioned in an optical path of light 350 returned by the scanning beam endoscope through the second connector interface. The image generation and display system is electrically coupled with an output of the photodetector and may generate and display images based on the backscattered light detected at different points in time during the scan. Alternatively, the photodetector may be located outside of the base station, such as, for example, in the scanning beam endoscope. As another potential variation, the display may optionally be included separate from the base station and attachable to the base station.

The first base station has a first communication interface 334. Likewise, the second base station has a second communication interface 342. The communication interfaces are communicatively coupled through a communication link 336. The communication link is commonly an electrical cable, or other electrical communication link, although optical and wireless communication links are also suitable. The communication interfaces and communication link may allow synchronization or coordination signals to be exchanged, in either or both directions, between the base stations. Numerous types of links, such as, for example, coaxial cable, twisted pair, and the like, are suitable. The scope of the invention is not limited to any known type of communication link.

The first base station has a first controller 335. Likewise, the second base station has a second controller 341. The controllers may be implemented in hardware (e.g., a microcontroller), software (e.g., a program or routine), or a combination of hardware and software. Either or both of the controllers may be capable of generating the coordination signals. In embodiments of the invention, either or both of the controllers may have, or may be in communication with, a clock, crystal oscillator, or other time-keeping circuit or device. The controller may use the time-keeping circuit or device to time or synchronize the generation and exchange of the coordination signals over the communication interfaces and link. In one aspect, the coordination signals may be exchanged repeatedly, for example multiple times a second, according to a desired frame rate for each of the endoscope systems. In one aspect, this may be performed continually while both endoscope systems are used for imaging.

The first controller 335 is electrically coupled with, or otherwise in communication with, the first communication interface 334. The first controller is also electrically coupled with, or otherwise in communication with, the first light source 331. The first controller may be operable to reduce or turn off an intensity of the light provided by the first light source at coordinated or synchronized times that are based on the exchange of the coordination signals. The first controller may know or become aware of the exchange of the coordination signal. For example, the first controller may generate the coordination signal, or cause the coordination signal to be transmitted. As another example, the first controller may receive the coordination signal, or at least indication of the exchange of the coordination signal. In conjunction with the exchange of the coordination signal, the first controller may provide a control signal to the first light source to reduce or turn off the intensity of the light provided by the first light source.

This may reduce or stop the illumination of the surface with the light from the first endoscope. As shown by dashed lines in the illustration, the light from the first endoscope system may be turned off, or at least reduced in intensity, while the scanning beam endoscope system is acquiring an image of the surface. As a result, at least less backscattered light 358 from the first endoscope system may be collected by the scanning beam endoscope. Note that the intensity of the light from the first endoscope system may remain reduced or turned off throughout the acquisition of the image by the scanning beam endoscope.

The second controller 341 is electrically coupled with, or otherwise in communication with, the second communication interface 342. In embodiments of the invention, the second controller may be operable to cause the display of images acquired by the scanning beam endoscope at coordinated or synchronized times that are based on the exchange of the coordination signals. There are different ways in which this may be done.

As a first example approach, according to embodiments of the invention, the scanning beam endoscope system may only acquire images after and while the intensity of the light has been reduced. The scanning beam endoscope system may not be in the process of or may omit acquiring an image of the surface while the surface is illuminated with the full intensity of light from the first endoscope system. Rather, in embodiments of the invention, the second controller may control the scanning beam endoscope system to begin to acquire the image of the surface only after the intensity of the light has been reduced.

Certain scanning beam endoscopes may have the capability of interrupted resonance. Such scanning beam endoscopes are capable of waiting idle, for various or arbitrary periods of time, until a coordination signal is exchanged. Then, responsive to the exchange of the coordination signal, the scanning beam endoscope may begin on demand to acquire an image. For example, a cantilevered optical fiber may be kept substantially motionless while the surface is illuminated with the full intensity of light from the first endoscope system. Then the cantilevered optical fiber may begin to be vibrated after the intensity of the light has been reduced.

If a shorter period of time is available for the scan, the scanning beam endoscope may perform a faster scan. Otherwise, if a longer period of time is available for the scan, the scanning beam endoscope may perform a slower scan. In one or more embodiments of the invention, the coordination signal may optionally specify a length of time (e.g., a length of time available to perform the scan while the light from the other endoscope is reduced or turned off), although this is not required. The scanning beam endoscope system may acquire an image over a length of time that is shorter than, equal to, not longer than, or otherwise based on the specified length of time. Further background information on interrupted resonance, if desired, is available in U.S. Published application Ser. No. ______, entitled “SCANNING BEAM WITH VARIABLE SEQUENTIAL FRAMING USING INTERRUPTED SCANNING RESONANCE,” by Richard S. Johnston et al.

As shown, the second controller 341 may be electrically coupled with, or otherwise in communication with, each of the second light source 344 and the actuator driver 343. In one or more embodiments of the invention, the second controller may, at coordinated times that are based on the exchange of the coordination signals, provide control signals to the second light source and the actuator driver to turn the second light source on and cause the actuator driver to begin to provide actuator drive signals to the scanning beam endoscope through the second connector interface. In this approach, actuator drive signals may not be provided to the connector interface at times other than the coordinated times. Alternatively, the second light source may remain on, in which case the control signal to the second light source is not needed. In embodiments of the invention, the control signals may be provided in conjunction with, for example responsive to, and/or immediately after, the exchange of a coordination signal.

The control signals may cause the scanning beam endoscope to begin to acquire an image of the surface. For example, the light provided through the second connector interface may be emitted as a beam or illumination spot from the scanning beam endoscope. The actuator drive signals may cause the scanning beam endoscope to scan the beam or illumination spot over the surface in a spiral, propeller, raster, or other suitable scan pattern. For example, the actuator drive signals may cause a piezoelectric tube actuator to begin to vibrate a single cantilevered optical fiber from rest to close to or within a Q-factor of its resonant frequency.

As a second example approach, rather than the scanning beam endoscope only acquiring images while the intensity of the light from the first endoscope system has been reduced, the scanning beam endoscope system may continually acquire images, including at times when the surface is illuminated with the full intensity of the light from the first endoscope. However, rather than displaying the images acquired when the light is at full intensity, the second controller may cause these images to be selectively discarded and/or the display of these images to be selectively omitted, since these images are generally of lower contrast or quality than the images acquired once the intensity of the light is reduced. Although discarding these images may reduce the frame rate of the scanning beam endoscope system, the images displayed may be of higher quality. In one or more embodiments, the most recently captured high quality image may be displayed on the screen until another high quality image is available to prevent the screen from going blank.

As shown, in embodiments of the invention, the second controller 341 may optionally be electrically coupled with, or otherwise in communication with, the image generation and display system 346. In embodiments of the invention, the second controller may, at coordinated times based on the exchange of the coordination signals, provide one or more control signals to the image generation and display system. The control signals may cause an image acquired while the intensity of the light is reduced to be displayed and/or may cause an omission of the display of an image acquired while the surface is illuminated with the full intensity of the light. As one example, the control signals may selectively turn the image generation and display system on while the intensity of the light is reduced. As another example, the control signals may selectively turn the image generation and display system off while the surface is illuminated with the full intensity of the light. Alternatively, other approaches for omitting displaying images, such as flushing a display buffer, or overwriting the display buffer with the previously acquired high quality image, are also possible.

After acquiring the image with the scanning beam endoscope system, in embodiments of the invention, the second controller may at coordinated times optionally provide one or more control signals to turn off or at least reduce an intensity of the light from the second light source, although this is not required. In embodiments of the invention, this may be in conjunction with the exchange of another coordination signal. Advantageously, this may potentially help to improve the quality of images acquired using the first endoscope system.

Another approach for coordinating image acquisition among multiple endoscope systems may involve the scanning beam endoscope system sensing or otherwise detecting the reduction of the intensity of the light from the first endoscope system.

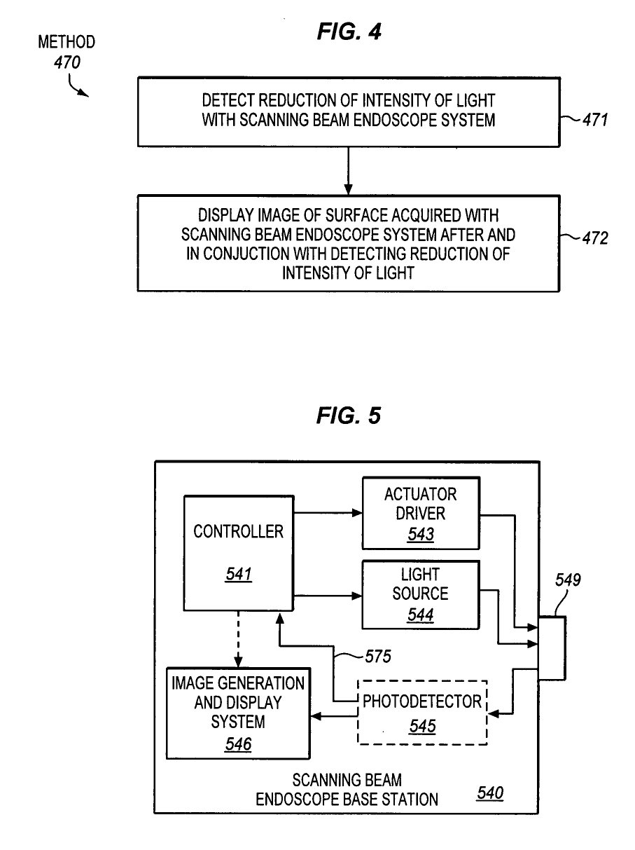

FIG. 4 is a block flow diagram of a method 470 of coordinating image acquisition among a first endoscope system and a second scanning beam endoscope system, according to embodiments of the invention. At block 471, a reduction of intensity of light from the first endoscope system may be detected with the scanning beam endoscope system. There are different ways of detecting the reduction of the intensity.

In one approach, light backscattered from a surface of which an image is to be acquired, may be collected with the scanning beam endoscope. The intensity of the collected light may be detected, for example, with one or more photodetectors. The detected intensity may be compared with a threshold intensity. The value of the threshold intensity may be selected to be greater than the intensity that would be detected once the intensity of the light from the first endoscope system has been reduced, and less than the intensity that would be detected if the light from the first endoscope system had not yet been reduced. If the detected intensity is determined to be less than the threshold intensity, then it may be inferred that the intensity of the light from the first endoscope system has already been reduced or turned off. Conversely, if the detected intensity is determined to be greater than the threshold intensity, then it may be inferred that the intensity of the light from the first endoscope system has not yet been reduced. To avoid missing a reduction cycle, generally the frequency of detection and comparison with the threshold should be greater than the frequency at which the light from the first endoscope system is reduced.

In another approach, light may be collected and detected by the scanning beam endoscope at different times. Then, rather than comparing the detected intensities to a threshold intensity, the intensities detected in sequential times may be compared with one another. If a difference between an intensity and a subsequently detected intensity is positive and the absolute value of the difference is sufficiently great, for example greater than a difference threshold, then it may be inferred that the intensity of the light from the first endoscope system has already been reduced or turned off. Conversely, if the difference between an intensity and a subsequently detected intensity is negative and the absolute value of the difference is sufficiently great, for example greater than a difference threshold, then it may be inferred that the intensity of the light from the first endoscope system has been increased again. If the difference is not sufficiently great, it may be inferred that the intensity has not been reduced or increased.

Referring again to FIG. 4, an image of the surface acquired with the scanning beam endoscope system after and in conjunction with detecting the reduction of the intensity of the light from the first endoscope system may be displayed, at block 472. In one or more embodiments of the invention, the scanning beam endoscope system may selectively start to acquire the image in conjunction with detecting the reduction, for example using the aforementioned technique of interrupted resonance. Alternatively, in one or more embodiments of the invention, the scanning beam endoscope system may selectively display the image acquired after and in conjunction with detecting the reduction, while selectively omitting the display of an image acquired immediately before the reduction is detected.

FIG. 5 is a block diagram of one example of a scanning beam endoscope base station 540 having at least one optional photodetector 545 and a controller 541 to coordinate image acquisition with another endoscope system by detecting a reduction of intensity of the light from the other endoscope system, according to embodiments of the invention.

The base station includes the controller 541, an actuator driver 543, a light source 544, a connector interface 549, the photodetector 545, and an image generation and display system 546. For brevity, and unless otherwise specified, these components may optionally have some or all of the characteristics of the correspondingly named components of the scanning beam endoscope base station of FIG. 3.

In the illustrated base station, the at least one optional photodetector is included in the base station, although this is not required. The photodetector may detect the intensity of light collected by the scanning beam endoscope and returned to the base station through the connector interface. The photodetector is electrically coupled with, or otherwise in communication with, the controller through a communication link 575. The photodetector may provide the detected intensities to the controller. Alternatively, the photodetector may be located outside of the base station, such as, for example, in the scanning beam endoscope. The photodetector in the endoscope may be in communication with the controller of the base station through a signal path through the connector interface to provide the detected intensities to the controller.

The controller may be operable to detect the reduction of the intensity of light using one or more of the approaches discussed above. The controller may also be operable to cause display of images acquired by the scanning beam endoscope at coordinated times that are based in part on the detected intensities. In one or more interrupted resonance embodiments of the invention, the controller may selectively provide one or more control signals to the actuator driver to cause the actuator driver to selectively begin to provide actuator drive signals to the scanning beam endoscope through the connector interface. Alternatively, in one or more embodiments, the controller may provide one or more control signals to the image generation and display system to selectively display an image acquired while the intensity is reduced and/or selectively omit displaying an image acquired before the intensity is reduced.

Advantageously, there is no need for a dedicated communication link between the scanning beam endoscope system and the first endoscope system to allow them to coordinate image acquisition. Rather, the endoscope systems may “communicate” indirectly through the scanning beam endoscope detecting the changing amount of light provided by the first endoscope system. In a broad sense, the optical signal exchanged between the first endoscope system and the photodetector of the scanning beam endoscope system may be considered a coordination signal exchanged between the endoscope systems to coordinate image acquisition. In a broad sense, the previously described coordination signal and the presently described optical signals may each be considered an impetus or stimulus from the first endoscope system to incite the scanning beam endoscope system into coordinated image acquisition.

A related approach, which may optionally be used with the approaches described herein, is described in co-pending U.S. patent application Publication Ser. No. ______, entitled “IMPROVED IMAGE ACQUISITION THROUGH FILTERING IN MULTIPLE ENDOSCOPE SYSTEMS”, filed on ______, by Richard S. Johnston.

In the description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other.

In the description above, for the purposes of explanation, numerous specific details have been set forth in order to provide a thorough understanding of the embodiments of the invention. The particular embodiments described are not provided to limit the invention but to illustrate it. Embodiments may be practiced without some of these specific details. Furthermore, modifications may be made to the embodiments disclosed herein, such as, for example, to the configurations, functions, and manner of operation, and use, of the components. All equivalent relationships to those illustrated in the drawings and described in the specification are encompassed within embodiments of the invention. The scope of the invention is not to be determined by the specific examples provided above but by the claims below. Further, where considered appropriate, reference numerals or terminal portions of reference numerals may have been repeated among the figures to indicate corresponding or analogous elements, which may optionally have similar characteristics.

It should also be appreciated that reference throughout this specification to “one embodiment,” “an embodiment,” or “one or more embodiments,” for example, means that a particular feature may be included in the practice of the invention. Similarly, it should be appreciated that in the description various features are sometimes grouped together in a single embodiment, Figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. A method comprising:
acquiring an image of a surface with a first endoscope system by illuminating the surface with light, collecting backscattered light, and generating the image based on the collected backscattered light;
reducing an intensity of the light from the first endoscope system; and
while the intensity of the light is reduced, acquiring an image of the surface with a second, scanning beam endoscope system by scanning a beam of light over the surface and collecting backscattered light at different times while the beam is scanned over the surface.

2. The method of claim 1, further comprising exchanging a coordination signal over a communication link between the first endoscope system and the scanning beam endoscope system, and wherein the intensity of the light is reduced in conjunction with the exchange of the coordination signal.

3. The method of claim 2, wherein exchanging the coordination signal comprises exchanging a coordination signal that specifies a length of time, and wherein acquiring the image with the scanning beam endoscope system comprises acquiring the image over a length of time that is based on the specified length of time.

4. The method of claim 1, further comprising detecting the reduction of the intensity of the light with the scanning beam endoscope system.

5. The method of claim 4, wherein detecting the reduction comprises:
detecting an intensity of light backscattered from the surface with the scanning beam endoscope after reducing the intensity of the light from the first endoscope system;
comparing the detected intensity with a threshold intensity; and
determining that the detected intensity is less than the threshold intensity.

6. The method of claim 4, wherein acquiring the image with the scanning beam endoscope system comprises starting to acquire the image in conjunction with detecting the reduction of the intensity.

7. The method of claim 1, further comprising omitting acquiring an image of the surface with the scanning beam endoscope system while the surface is illuminated with the light from the first endoscope system.

8. The method of claim 1, further comprising:
keeping a cantilevered optical fiber of the scanning beam endoscope system substantially motionless while the surface is illuminated with the light from the first endoscope system; and
beginning to vibrate the cantilevered optical fiber after the intensity of the light from the first endoscope system has been reduced.

9. The method of claim 1, further comprising acquiring an image of the surface with the scanning beam endoscope system while the surface is illuminated with the light from the first endoscope system.

10. The method of claim 9, further comprising:
displaying the image acquired while the intensity of the light is reduced; and
omitting displaying the image acquired while the surface is illuminated with the light from the first endoscope system.

11. The method of claim 1, wherein reducing the intensity of the light comprises turning off the light from the first endoscope system.

12. The method of claim 1, further comprising, after acquiring the image with the scanning beam endoscope system, reducing an intensity of the beam of light.

13. The method of claim 1, repeatedly performed multiple times a second for at least several consecutive seconds.

14. The method of claim 1, wherein acquiring the image of the surface with the scanning beam endoscope system comprises vibrating a single cantilevered optical fiber within a Q-factor of its resonant frequency.

15. An apparatus comprising:
a connector interface to allow an endoscope to be connected;
a light source to provide light to the endoscope through the connector interface;
a communication interface to an endoscope system, the communication interface to allow coordination signals to be exchanged with the endoscope system;
a controller in communication with the communication interface, the controller operable to cause display of images acquired by the endoscope at coordinated times that are based on the exchange of the coordination signals.

16. The apparatus of claim 15, wherein the controller is operable to cause the apparatus to omit acquiring images with the endoscope except at the coordinated times.

17. The apparatus of claim 16, further comprising an actuator driver in communication with the controller, wherein the controller is operable to cause the actuator driver to provide actuator drive signals to the connector interface at the coordinated times without providing actuator drive signals to the connector interface at times other than the coordinated times.

18. The apparatus of claim 15, wherein the controller is operable to cause the apparatus to omit displaying images acquired by the endoscope at times other than the coordinated times.

19. The apparatus of claim 15, wherein the controller is operable to generate a coordination signal that specifies a length of time.

20. The apparatus of claim 15, wherein the controller is operable to cause a reduction in an intensity of the light from the light source at a time other than the coordinated times.

21. An apparatus comprising:
a connector interface to allow an endoscope to be connected;
a light source to provide light to the endoscope through the connector interface;
a communication interface to an endoscope system, the communication interface to allow coordination signals to be exchanged with the endoscope system;
a controller in communication with the communication interface, the controller operable to reduce an intensity of the light provided by the light source at coordinated times that are based on the exchange of the coordination signals.

22. The apparatus of claim 21, wherein controller is operable to turn off the light provided by the light source based on the exchange of the coordination signals.

23. The apparatus of claim 21, wherein the controller is operable to generate a coordination signal that specifies a length of time.

24. The apparatus of claim 21; wherein the light source comprises a broad bandwidth light source having a bandwidth of at least 200 nm.

25. The apparatus of claim 21, wherein the light source comprises at least one narrow bandwidth light source having a bandwidth of less than 3 nm.

26. An apparatus comprising:
a connector interface to allow an endoscope to be connected;
a light source to provide light to the endoscope through the connector interface;
a controller operable to cause display of images acquired by the endoscope at coordinated times that are based in part on intensities of light detected by at least one photodetector.

27. The apparatus of claim 26, further comprising the at least one photodetector.

28. The apparatus of claim 26, further comprising a signal path through the connector interface to receive the detected intensities from the at least one photodetector.

29. The apparatus of claim 26, wherein the controller is to compare the detected intensities or differences between two or more of the detected intensities with a threshold intensity.
